SEQUENCE LISTING (10 sequences)

SEQ ID NO: 1 - protein, 104 aa, Artificial
  Synthetic protein
GPPGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGG

SEQ ID NO: 2 - protein, 401 aa, Artificial
  Synthetic protein
GPPGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGG

SEQ ID NO: 3 - protein, 99 aa, Artificial
  Synthetic protein
GEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPA

SEQ ID NO: 4 - protein, 396 aa, Artificial
  Synthetic protein
GEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPA

SEQ ID NO: 5 - protein, 792 aa, Artificial
  Synthetic protein
GEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPA

SEQ ID NO: 6 - protein, 1188 aa, Artificial
  Synthetic protein
GEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPA

SEQ ID NO: 7 - protein, 1584 aa, Artificial
  Synthetic protein
GEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPA

SEQ ID NO: 8 - protein, 797 aa, Artificial
  Synthetic protein
GPPGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGG

SEQ ID NO: 9 - protein, 1193 aa, Artificial
  Synthetic protein
GPPGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGG

SEQ ID NO: 10 - protein, 1589 aa, Artificial
  Synthetic protein
GPPGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGEPGNPGSPGNQGQPGNKGSPGNPGQPGNEGQPGQPGQNGQPGEPGSNGPQGSQGNPGKNGQPGSPGSQGSPGNQGSPGQPGNPGQPGEQGKPGNQGPAGG

(19) United States
(12) Patent Application Publication
(10) Pub. No.: US 2010/0256075 A1
(43) Pub. Date: Oct. 7, 2010

(54) High Yield Secretion of Multimeric Recombinant Protein

(76) Inventors: Arjo Lysander De Boer, Holland (NL); Hendrik Van Urk, Holland (NL); Jan Bastiaan Bouwstra, Holland (NL); Peter Franciscus Theresius Maria Van Asten, Holland (NL)

Correspondence Address: MORGAN LEWIS & BOCKIUS LLP, 1111 PENNSYLVANIA AVENUE NW, WASHINGTON, DC, 20004, US

(21) Appl. No.: 12/678,065

(22) PCT Filed: Sep. 9, 2008
(86) PCT No.: PCT/NL2008/050593

§ 371 Date: Mar. 12, 2010

(30) Foreign Application Priority Data
Sep. 14, 2007 (EP) 07116494.1

(51) Int. Cl. A61K 38/16; C07K 14/00; C12P 21/00; A61P 29/00; A61P 35/00; A61P 9/00; A61P 19/08; A61P 25/00; A61P 3/10

(52) U.S. Cl. 514/21.2; 530/350; 435/69.1

(57) ABSTRACT

The present invention relates to high molecular weight gelatin-like proteins and methods for producing high yields thereof. Also, compositions comprising one or more of such proteins are provided.

### FIELD OF THE INVENTION

The present invention relates to the field of gene expression systems and the production of recombinant proteins or polypeptides. Provided are methods for producing high yields of high molecular weight gelatins (40 kDa and larger), as well as the gelatins as such and compositions comprising one or more of these or consisting of one or more of these.

### BACKGROUND OF THE INVENTION

Werten et at (2001, Protein Engineering 14:447-454) describe the production of a recombinant polar (hydrophilic) gelatin-like polypeptide in Pichia pastoris. Both a monomeric unit (P) (SEQ ID NO: 1) and a tetrameric unit (P4) (SEQ ID NO 2) are expressed. The yield of the tetrameric (P4) was found to be 3-6 g/l of clarified broth. The P4 polypeptide was non-hydroxylated and had an open structure (i.e. did not form triple helices at 4° C. and was essentially non-gelling). Also, the high polarity accounted for negligible surface activity in water at concentrations of up to 5% (w/v) as determined by tensiometry.

EP 0926543 describes the production of non-hydroxylated, recombinant mouse type I (Col1A1, 28 kDa and 53 kDa) and rat type III (Col3A1, 21 kDa) collagen-like polypeptides in Pichia pastoris at a yield of 2-3 g/l for single copy tranformants, with up to 14.8 g/liter clarified broth of multicopy transformants (Werten et al. 1999, Yeast 15, 1087-1096). The polypeptides used were fragments of natural Col1A1 and Col1A3, whereby the fragments were part of the triple helix domain, comprising Gly-Xaa-Yaa triplets.

Many problems exist with instability of highly repetitive synthetic genes (Cappello, 1990, Trends Biotechnol. 8, 309-311). Native gelatin sequences may be more stable than synthetic sequences, due to their higher variability in amino acid sequence.

There remains a need for producing high yields of recombinant, synthetic collagen-like proteins or polypeptides, especially synthetic sequences and/or non-gelling (non-hydroxylated) sequences. High molecular weight sequences (such as polypeptides of calculated molecular weights of 70 kDa or more) are particularly difficult to produce at high yields, as degradation problems are more problematic than for low molecular weight polypeptides.

### SUMMARY OF THE INVENTION

In the search for further improvements of the yields at the production of recombinant gelatins which might be suitable for various applications, the present inventors surprisingly found that upon trying to recombinantly produce gelatins with high molecular weight, the yield of the polypeptide obtained was unexpectedly high, in fact even better than for similar, yet smaller polypeptides. A multimer polypeptide was identified consisting of or comprising at least 5 consecutive repeat units of a monomer polypeptide unit, wherein said monomer polypeptide unit comprises at least 30 consecutive Gly-Xaa-Yaa triplets, wherein Gly is Glycine and Xaa and Yaa are any amino acid. In one embodiment of the invention, the recombinant gelatins are provided, as well as pharmaceutical or nutraceutical compositions or cell supports comprising the recombinant gelatins. Also methods for using the recombinant gelatins and/or the cell supports or controlled release compositions for cell adhesion related medical applications are provided.

### GENERAL DEFINITIONS

“Gelatin” and “gelatin-like” and “collagen” and “collagen-like” proteins or polypeptides are used herein interchangeably to refer to amino acid chains comprising or consisting of Gly-Xaa-Yaa (GXY) repeats. Also, the terms “gelatin”, “protein”, “peptide” and “polypeptide” are used interchangeably herein.

“High molecular weight” refers herein to polypeptides of at least about 40 kDa calculated molecular weight, such as polypeptides of equal to or above about 50, 60 and in particular of equal to or above about 70, 80, 90, 100, 110, 120, 130, 140, 150, 200, 250 up to 300 kDa, or more.

The term “substantially identical”, “substantial identity” or “essentially similar” or “essential similarity” means that two polypeptide, when aligned pairwise using the Smith-Waterman algorithm using default parameters. comprise at least 70%, 72%, 74%, 75%, 76%, 77% or 78%, preferably at least 80%, more preferably at least 85%, 90%, 95%, 98%. 9 or more amino acid sequence identity. Moree preferably, the polypeptides comprise said amino acid sequence identity while having no more than 3 gaps, preferably no more than 2 gaps, even more preferably no more than 1 gap and most preferably 0 gaps in the alignment. Sequence alignments and scores for percentage sequence identity may be determined using computer programs, such as the GCG Wisconsin Package, Version 10,3, available from Accelrys Inc., 9685 Scranton Road, San Diego, Calif. 92121-3752 USA or using in EmbossWIN (e.g. version 2.10.0). For comparing sequence identity between two sequences, it is preferred that local alignment algorithms are used, such as the Smith Waterman algorithm (Smith T F, Waterman M S (1981) J. Mol. Biol 147(0;195-7), used e.g. in the EmbossWlN program “water”. Default parameters are gap opening penalty 10.0 and gap extension penalty 0.5, using the Blosum62 substitution matrix for proteins (Henikoff & Henikoff, 1992, PNAS 89, 915-919).

The term “comprising” is to be interpreted as specifying the presence of the stated parts, steps or components, but does not exclude the presence of one or more additional parts, steps or components.

In addition, reference to an dement by the indefinite article “a” or “an” does not exclude the possibility that more than one of the element is present, unless the context clearly requires that there be one and only one of the elements. The indefinite article “a” or “an” thus usually means “at least one”.

“Monomer” refers to a polypeptide unit which can be used to generate a “multimer” by repeating the unit in a linear fashion to generate a longer polypeptide. The monomer units are preferably repeated without intervening amino acids, although optionally 1, 2, 3, 4 or 5 linking amino acids may be present between monomer units. Polypeptide with (SEQ ID NO: 3) herein is an example of a monomer. Polypeptide with (SEQ ID NO: 4) herein is a “repeat with molecular weight below 70 kDa”, in particular the “repeat” is a tetramer, of the repeat unit of monomer “P” (SEQ ID NO: 3). The repeat unit of Polypeptide “P4” (SEQ ID NO: 4) may be repeated 2, 3, 4 times or more to form multimers of 8 P repeat units (8-mer) (SEQ ID NO: 5), 12 P repeat units (12-mer) (SEQ ID NO: 6), 16 P repeat units (16-mer) (SEQ ID NO: 7), etc.

“Host” or “host organism” or “recombinant host cell” refers herein to the microorganism into which the nucleic acid sequence encoding the polypeptide according to the invention is introduced. Preferred hosts are yeasts of the genus Pichia (preferably Pichia pastoris, Hansenula (preferably Hansenula polymorpha), Axula (preferably Axula adeninivorans)

P (SEQ ID NO: 1):
GPP GEP GNP GSP GNQ GQP GNK GSP GNP GQP GNE GQP
GQP GQN GQP GEP GSN GPQ GSQ GNP GKN GQP GSP
GSQ GSP GNQ GSP GQP GNP GQP GEQ GKP GNQ GPA GG
P4 (SEQ ID NO: 2):
GPP GEP GNP GSP GNQ GQP GNK GSP GNP GQP GNE GQP
GQP GQN GQP GEP GSN GPQ GSQ GNP GKN GQP GSP
GSQ GSP GNQ GSP GQP GNP GQP GEQ GKP GNQ GPA
GEP GNP GSP GNQ GQP GNK GSP GNP GQP GNE GQP
GQP GQN GQP GEP GSN GPQ GSQ GNP GKN GQP GSP
GSQ GSP GNQ GSP GQP GNP GQP GEQ GKP GNQ GPA
GEP GNP GSP GNQ GQP GNK GSP GNP GQP GNE GQP
GQP GQN GQP GEP GSN GPQ GSQ GNP GKN GQP GSP
GSQ GSP GNQ GSP GQP GNP GQP GEQ GKP GNQ GPA
GEP GNP GSP GNQ GQP GNK GSP GNP GQP GNE GQP
GQP GQN GQP GEP GSN GPQ GSQ GNP GKN GQP GSP
GSQ GSP GNQ GSP GQP GNP GQP GEQ GKP GNQ GPA GG.

### DETAILED DESCRIPTION

### Polypeptides and Nucleic Acid Sequences According to the Invention

In one aspect of the invention gelatin-like polypeptides are provided, which have a high molecular weight. Gelatin-like polypeptides preferably comprise at least a region of 15, 20, 25, 30, 33, 35, 40, 45, 50 or more consecutive GXY triplets (the monomer unit), which is preferably repeated at least 5, 6, 7, 8, 9, 10, 11, 12, 13, 14, 15, 16, 17, 18, 19 or 20 times to form a high molecular weight polypeptide. In a preferred embodiment the monomer unit is repeated with an even number of repeats, i.e. the high molecular weight polypeptide comprises at least 8, 10, 12, 14, 16, 18, 20, 24, 28 or 32 times the monomer unit.

In the GXY triplets, X (also Xaa) and Y (also Yaa) may be any amino acid. In natural collagen Xaa and Yaa are often proline and hydroxyproline, respectively, with the hydroxyproline being hydroxylated posttranslationally, e,g. by a prolyl-4-hydroxylase present in the host cell. Herein, the polypeptides according to the invention are preferably essentially free of posttranslational modifications by prolyl-4-hydroxylase (P4H) enzymes, as they are produced in recombinant host cells, such a the methylotrophic yeast Pichia, into which no heterologous genes encoding a functional P4H enzyme have been introduced. Thus, in one aspect of the invention, the monomer and multimer are free of hydroxyproline and free of triple helix structure characteristic of natural collagen. “Free of hydroxyproline” refers herein to gelatin-like polypeptides that are in essence free of hydroxyproline residues, meaning that less than 2% of the amino acid residues in the gelatin-like protein are hydroxyproline residues, preferably less than 1%, more preferably no hydroxyproline residues are present. The amount of hydroxyprolines can be determined by any standard amino acid analysis method like, for example, described in HP AminoQuant Series II, operators handbook, 1990, Hewlett-Packard GmbH, Federal Republic of Germany, Waldbronn Analytical Division, HP Part No. 01090-90025.

“Free of triple helix” structure refers to essentially the absence of the positive peak characteristic of the collagen triple helix in a circular dichroism spectrum. Circular dichroism spectrometry can be carried out as described in Werten et at (2001, Protein Engineering 14:447-454).

In one aspect of the invention the polypeptides are more hydrophilic than natural gelatin. For example, the monomer and/or multimer has a GRAVY value (Grand average of hydrophilicity; Kyte and Doolittle 1982, J. Mol. Biol. 157, 105-132) of less then −1.4, such as less than or equal to −1.5, −1.6, −1.7, −1.8, −1.9, etc. Hydrophilicity can be increased by reducing the percentage of hydrophobic amino acids in the sequence (such as Trp, Tyr, Phe, Leu, Ile, Val and Met). E.g. the monomer and/or multimer polypeptides may comprise less than 3, 2, or 1, most preferably 0 of the mentioned hydrophobic amino acids, other than Proline and Glycine. Also, the monomer and/or multimer may comprise a high amount of hydrophilic amino acids, such as Asparagine Asn) and/or Glutamine (Gln).

Although short intervening amino acids or stretches of amino acids may be present between the repeat units, it is preferred that the repeats are essentially free of intervening amino acids, whereby “essentially free” means that less than 5, 4, 3, 2 or I, most preferably 0 intervening amino acids are present between monomers. The multimer may comprise additional amino acids at one or both ends, e.g. at the N- and/or C-terminal. For example, 1, 2, 3, 6, 9, 12, 15 or more amino acids may be present. These may be in the form of GXY triads.

In order to facilitate multimer construction, the multimeric protein may comprise N-terminal and C-terminal amino acids that are not part of the repeating amino acid sequence.

The monomer unit may be a fragment of a natural collagen protein (such as human type I, II or III collagen proteins, e.g. Col1A1, Col3A1, etc.) but is preferably a synthetic sequence, not occurring in nature.

In one embodiment the monomer comprises or consists of SEQ ID NO: 3 (P repeat unit) or an amino acid sequence essentially identical thereto. An amino acid sequence essentially identical to SEQ ID NO: 3 is an amino acid sequence which comprises at least 70%, 80%, 85%, 89%, 90%, 95%, 98%, 99% or more amino acid identity to SEQ ID NO: 3, when aligned pairwise using the Smith Waterman algorithm, with default parameters as defined above.

In one embodiment the repeat with molecular weight below 70 kDa comprises or consists of SEQ ID NO: 4 (P4 repeat unit) or an amino acid sequence essentially identical thereto. An amino acid sequence essentially identical to SEQ ID NO: 4 is an amino acid sequence which comprises at least 70%. 80%, 85%, 89%, 90%, 95%, 98%, 99% or more amino acid identity to SEQ ID NO: 4, when aligned pairwise using the Smith Waterman algorithm, with default parameters as defined above.

The monomer and repeat nucleic acid sequences are preferably made using known molecular biology techniques, e.g. from de novo synthesis or by cloning fragments of natural collagen like proteins and optionally further DNA modification to encode the desired amino acids. Once a nucleic acid sequence encoding the monomer is made, standard cloning techniques can he used to repeat tms nucleic. acid sequence in a linear fashion in order to generate a nucleic acid sequence encoding the high molecular weight protein (An example can be found in Werten et al. (2001, Protein Engineering 14:447-454). Due to the degeneracy of the genetic code, obviously different nucleic acid molecules can encode the same amino acid sequence. The codon usage of the nucleic acid sequence is preferably adapted to the codon usage of genes which are highly expressed in the host (see Sreekrishna and Kropp, 1996, Nonconventional yeasts in biotechnology. A handbook. Springer, Berlin, p203-253).

Thus, the nucleic acid sequence encoding the monomer is preferably repeated consecutively, to form a nucleic acid sequence encoding a high molecular weight multimer, which can then be produced in a recombinant microorganism host as described herein below.

Preferred multimers are multimers of P repeat unit (monomer) and/or P4 repeat described above, or variants of these. Most preferably, the same monomer and/or repeat unit is repeated to form the high molecular weight polypeptide. In one embodiment P mer, P 12-mer and P 16-mer are provided herein, as depicted in SEQ ID NO: 5, 6, and 7, respectively, as well as variants thereof. Variants of SEQ ID NO: 5, 6 and 7 include polypeptides comprising or consisting of an amino acid sequence which comprises at least 70%, 80%, 85%, 89%, 90%, 95%, 98%, 99% or more amino acid identity to SEQ ID NO: 5, 6 or 7, when aligned pairwise using the Smith Waterman algorithm, with default parameters as defined above.

In one embodiment the multimer recombinant gelatins according to the present invention, e.g. SEQ ID NO 5, 6 and 7 are preceded by a glycine-proline-proline (GPP) triplet and extended with two glycine residues (GO) at the carboxy-terminus. Thus recombinant gelatins according to the present invention include GPP((SEQ ID NO: 3))ₓGG, wherein x is an integer selected of 5 and higher, preferably x is 8 or 12 or 16. Respectively these sequences are SEQ ID NO: 8, 9 and 10 and are preferred embodiments according to the present invention.

The high molecular weight proteins preferably have a calculated molecular weight of at least about 40, 50, 60, 70, 80, 90, 100, 120, 140, 180, 220, 260 up to about 300 or more kiloDaltons (kDa). The molecular weight can be calculated using computer programs such as EmbossWin pepstats. SDS-PAGE measured molecular weights may not allow a correct size estimation to be made. Preferably when referring to “a polypeptide” a plurality of polypeptides, of the same amino acid sequence and molecular weight are meant, i.e. a “homogenous” composition of proteins is referred to, unless stated otherwise herein. In certain embodiments also defined mixtures of two, three or more high molecular weight proteins are provided (see below).

### Methods of Producing High Molecular Weight Polypeptides According to the Invention

It was surprisingly found that by repeating monomer units, very high yields of high molecular weight proteins could be obtained. Without being bound by any theory, it is therefore thought that a direct correlation exists between the yield and molecular weight of multimeric proteins.

The high molecular weight multimer gelatines according to the invention can be produced by recombinant methods as disclosed in EP-A-0926543, EP-A-1014176 or WO01/34646. Also for enablement of the production and purification of gelatines of the invention reference is made to the examples in EP-A-0926543 and EP-A-1014176.

The polypeptides can be produced by expression of nucleic acid sequence encoding such polypeptides by a suitable micro-organism. The process can suitably be carried out with a fungal cell or a yeast cell. Suitably the host cell is a high expression host cell like Hansenula, Axula, Trichoderma, Aspergillus, Penicillium, Saccharomyces, Kluyveromyces, Neurospora or Pichia. Fungal and yeast cells are preferred to bacteria as they are less susceptible to improper expression of repetitive sequences. Most preferably the host will not have a high level of proteases that attack the collagen structure expressed. In this respect Pichia or Hansenula offers an example of a very suitable expression system. Use of Pichia pastoris as an expression system is disclosed in EP-A-0926543 and EP-A-1014176. in one embodiment the micro-organism is free of active post-translational processing mechanism such as in particular hydroxylation of proline and also hydroxylation of lysine. In another embodiment the host system has an endogenic proline hydroxylation activity by which the recombinant gelatine is hydroxylated. The selection of a suitable host cell from known industrial enzyme producing fungal host cells specifically yeast cells on the basis of the required parameters described herein rendering the host cell suitable for expression of recombinant gelatine-like proteins suitable in compositions according to the invention in combination with knowledge regarding the host cells and the sequence to be expressed will be possible by a person skilled in the art.

Also mutant host strains may be used, e.g. strains deficient in one or more proteolytic enzymes, although this is not necessary according to the present invention, as the recombinant polypeptides are highly stable and resistant to proteolysis.

In one embodiment a method for producing a high molecular weight multimer polypeptide, having a calculated molecular weight of at least 70 kDa, is provided comprising the steps of:
(a) generating an expression vector comprising a promoter operably linked to a nucleic acid sequence encoding a multimer polypeptide as described herein above;
(b) transforming a yeast species, preferably Pichia pastoris, with said expression vector;
(c) culturing said transformed yeast host under suitable conditions for producing said polypeptide;
(d) optionally purifying said polypeptide from the culture medium and/or the host cells,
   wherein said high molecular weight polypeptide is produced at a level of at least 10 g/l culture broth, preferably at least 12 g/l, more preferably at least 14 g/l.
   Compositions, Products and uses According to the Invention

In one embodiment the present inventions concerns a composition comprising at least one multimer according to the present invention. In one embodiment the composition is a pharmaceutical composition or a nutritional- or nutraceutical composition. For example the present multimers can be used as a plasma expander in blood substitute liquids. Also the present multimers can constitute or be comprised in a matrix for controlled drug release. Also the invention further provides use of a such controlled release composition for the preparation of a medicament for the treatment of pain, cancer therapy, cardiovascular diseases, myocardial repair, angiogenesis, bone repair and regeneration, wound treatment, neural stimulation/therapy or diabetics.

In another embodiment, the present invention concerns a solid support comprising at least one multimer according to the present invention. In one embodiment the solid support is a medical device, e.g. a stent, a cell support, a dermal filler and the like.

A cell support comprising a multimer according to the present invention may for example be selected from the group consisting of

1) a cell-culture support, such as a core bead (e.g. a microcarrier bead) or a Petri dish or the like, coated with one or more multimer polypeptides according to the present invention;

2) an implant or transplant device (such as hip-, dental-, or other implants, etc.) coated with one or more of the multimer polypeptides according to the present invention,

3) a scaffold or matrix for tissue engineering, such as artificial skin matrix material, coated with one or more multimer polypeptides according to the present invention;

4) a wound healing product coated with one or more multimer polypeptides according to the present invention

5) a tissue adhesive comprising or consisting of one or more multimer polypeptides according to the present invention.

Also the present recombinant proteins are highly useful in photographic applications, e,g. as protective colloid in silver halide emulsions. Also in one embodiment the present invention concerns a silver halide emulsions comprising a gelatin according to the present invention.

### Examples

The construction of pPIC9-P4, the vector comprising the gene for the tetramer of P, has been described in detail in Werten et al. (2001, Protein Engineering 14:447-454), which is incorporated by reference herein.

The BglII-Not fragment from pPIC9-P4 containing the AOXI promoter and the gene for P4 was subcloned from pPIC9-P4 into pPICZ A digested with the same enzymes to yield pPICZ-P4. The DraIII site in the Zeocin resistance gene from pPICZ-P4 was removed by site-directed mutagenesis to render the DraIII site in the gene for P4 unique. The HindIII-PflMI fragment containing the P4 gene from pPICZ-P4 was subcloned into pPICZ-P4 digested with Drain and HindIII.

This resulted in the formation of plasmid pPICZ-P8. Plasmid pPICZ-P12 was generated by subcloning the HindIII-PflMI fragment from pPICZ-P8 into pPICZ-P4-digested with HindIII and DraIII. By analogy, pPICZ-P16 was generated by subcloning the HindIII-PflMI fragment from pPICZ-P8 into pPICZ-P8-digested with HindIII and DraIII.

The plasmids pPICZ-P4, pPICZ-P8, pPICZ-P12 and pPICZ-P16 were linearized with PmeI and transformed into P. pastoris X-33. Multicopy integrants were selected on 1.0 and 1.5 mg/ml of Zeocin. Manufacturer's (Invitrogen) protocols were followed.

Representative strains resulting from these transformations were grown in high-density cell cultures under standard fermentation conditions (at a pH of about 4), and the yield of the relevant gelatins in the supernatants was determined using UPLC (using BSA as a standard).

Yields obtained were:

| | |
|---|---|
| P4 | 7-9 g/l |
| P8 | 14.19-15.89 g/l |
| P12 | 14.23-18.41 g/l |

1. A multimer polypeptide consisting of or comprising at least 5 consecutive repeat units of a monomer polypeptide unit, wherein said monomer polypeptide unit comprises at least 30 consecutive Gly-Xaa-Yaa triplets, wherein Gly is Glycine and Xaa and Yaa are any amino acid.

2. The multimer polypeptide according to claim 1, wherein said monomer unit consist of at least 33 consecutive Gly-Xaa-Yaa repeats.

3. The multimer polypeptide according to claim 1, wherein said monomer polypeptide unit consists of or comprises the amino acid sequence of SEQ ID NO: 3, or an amino acid sequence comprising at least 70% identity to SEQ ID NO: 3.

4. The multimer polypeptide according to claim 1, wherein said multimer polypeptide or comprises the amino acid sequence of SEQ ID NO: 5, 6, or 7, or a or an amino acid sequence comprising at least 70% identity to SEQ ID NO: 5, 6 or 7.

5. The multimer polypeptide according to claim 1, wherein said multimer polypeptide consists of the amino acid sequence of SEQ ID NO: 8, 9, or 10.

6. A composition comprising at least one multimer according claim 1.

7. The composition according to claim 6, wherein said composition is a pharmaceutical composition or a nutritional- or nutraceutical composition.

8. A solid support comprising at least one multimer according to claim 1.

9. The solid support according to claim 8, wherein the support is a medical device, a cell support or a dermal filler.

10. A method for producing a high molecular weight multimer having a calculated molecular weight of at least 40 kDa, comprising the steps of:
generating an expression vector comprising a promoter operably linked to a nucleic acid sequence encoding a multimer polypeptide according to any one of claims 1-5;
transforming a yeast species, preferably Pichia pastoris, with said expression vector;
culturing said transformed yeast host under suitable conditions for producing said polypeptide;
optionally purifying said polypeptide from the culture medium and/or the host cells, wherein said high molecular weight polypeptide is produced at a level of at least 10 g/l culture broth.

11. The method according to claim 10, wherein said multimer polypeptide comprises SEQ ID NO: 5, 6 or 7 or consists of SEQ ID NO: 8, 9, or 10.
